(19) United States
(12) Patent Application Publication
(10) Pub. No.: US 2004/0253249 A1
(43) Pub. Date: Dec. 16, 2004

(54) Pulsatile transdermally administered antigens and adjuvants

(76) Inventors: Edward M. Rudnic, Potomac, MD (US); Colin E. Rowlings, Potomac, MD (US); Beth A. Burnside, Bethesda, MD (US); Robert J. Guttendorf, Gaithersburg, MD (US)

Correspondence Address: Carella, Byrne, Bain, Gilfillan,, Cecchi, Stewart & Olstein, 5 Becker Farm Road, Roseland, NJ, 07068, US

(21) Appl. No.: 10/816,670

(22) Filed: Apr. 2, 2004

Related U.S. Application Data
(60) Provisional application No. 60/459,763, filed on Apr. 2, 2003.

(51) Int. Cl. A61K 39/00

(52) U.S. Cl. 424/184.1

(57) ABSTRACT

Methods for triggering immunogenic responses and for eliciting improved immunogenic responses to immunogens in humans or animals through pulsatile transdermal delivery of antigens and adjuvants to the Langerhans cells of the skin, are disclosed.

This application is a continuation of, and claims priority to, U.S. Provisional Application Ser. No. 60/459,763, filed on Apr. 2, 2003, the disclosures of which are herein incorporated by reference in their entireties.

### BACKGROUND

Vaccines enable the body to fight infection. In the developed world, most vaccines are delivered by injection. Some research is currently ongoing to develop vaccines and antigens for transdermal delivery in an effort to confer improved immunity as well as improved patient compliance. There are several advantages to delivering antigens transdermally. Transdermal administration may improve patient compliance because it eliminates the discomfort of injections and other problems associated with needle exchange. By pulsing the antigen one can realize a further advantage by reducing the irritation caused by long-term contact of the transdermal adhesive, which tends to be very irritating. Pulsatile delivery also improves the driving force of the antigen across the skin and creates a higher probability that the antigen will make contact with the correct recipient cell (Langerhans cells).

Numerous companies have focused on the delivery of vaccines in an effort to meet significant unmet clinical needs. For example IOMAI, Gaithersburg, Md., Cygnus, Vyteris, and others.

It is well known in the art that the Langerhans cells, located in the upper spinosum layer of the skin, are dendritic cells. Langerhans cells were described in 1868, and they play an important role in contact allergies, the rejection of skin transplants, and other immunological processes of the skin. It is also well known in the art that these cells are the outermost post of the immune system.

The method for improving an immune response is preferably one that induces improved resistance to infection post administration of the antigen, such as by inducing or elevating a T cell response to the pulsatile transdermal administered antigen. Pulsatile transdermal delivery has been well documented by Wong, et al WO 96/00111. The pulsatile transdermal administration of the antigen and adjuvant can be delivered in a pulsatile fashion making contact with the Langerhans cells. This in turn triggers the dormant T-helper cells and thus initiates a primary T-cell dependent immune response.

Often times an adjuvant must be delivered with the antigen concomitantly in order to elicit an immune response. In order to produce such an immune response the adjuvant is required to stimulate the Langerhans cells so that they recognize the antigen and are stimulated to mature into dendritic cells. Pulsatile delivery of the adjuvant prior to and throughout the transdermal delivery of the antigen would give a more complete immune response.

The pulsatile delivery of the adjuvant to the dendritic cells, or Langerhans cells, provides substantial advantages over traditional sustained style delivery of transdermal or patch technology. The benefit of dosing an adjuvant concomitantly with an immune response producing material in a pulsatile fashion is three fold. First, multiple dosage units or spacing the dosing of the agent should allow for improved absorption since the skin would not reach saturation during pulsatile delivery. Secondly, studies demonstrate that an increased frequency of a lower dose yields better results than bolus dosing. And thirdly, by pulsing the adjuvant into the skin there would be less local toxicity due not only to the adjuvants, but also the active agent used in the transdermal administration of the antigen.

Pulsatile transdermal dosing can be effected through the use of externally regulated mechanisms such as those listed, but are not limited to: mechanical means such as electrophoresis, phonophoresis, iontophoresis, gene gun, and others; and chemical means such as pH sensitive gels, swelling mechanisms, solubility dependent control and temperature. In fact, many of these mechanisms are listed in WO 96/00111 by Wong et al.

The distinct difference between the prior art of Wong and the applicant's invention is that the material is not delivered through the tissue, which in effect gives biologically significant levels of the material in the systemic circulation, but is rather just delivered to the Langerhans cells, which then carry the antigenic material to elicit an immunological response.

### Definitions

As used herein the term “antigenic material” shall mean any material which confers an immunologic response either in vitro or in vivo

As used herein the term “immunogen” shall mean any antigenic material

As used herein the terms “pulsatile” or “pulasatile fashion” shall describe the delivery of antigenic material such that a portion of the dose of the product is released, followed by a second portion of the dose of product, followed by a third portion of the dose of product, and so on.

### DESCRIPTION

Antigens of interest

The types of infections that may be prevented by vaccination are numerous. Included in this application is just a short list of example diseases treatable by pulsatile transdermal vaccine treatment. Possibilities include, but are not limited to: staph, fungal infections that arise from psoriasis, other topical bacterial infections, melanoma, salmonella, influenza, travelers' diarrhea, tetanus, and H. Pylori. In addition, various autoimmune diseases, allergies, and multiple cancers are also subject to pulsatile transdermal vaccine treatment.

The present invention covers therapeutic proteins which include but are not limited to allergenic proteins and digested fragments thereof. These include pollen allergens from ragweek, rye, June grass, orchard grass, sweet vernal grass, red top grass, timothy grass, yellow dock, wheat, corn, sagebrush, blue grass, California annual grass, pigweek, Bermuda grass, Russian thistle, mountain cedar, oak, box elder, sycamore, maple, elm, etc. dust and mites, bee venom, food allergens, animal dander, and other insect venoms.

Therapeutic proteins include microbial vaccines which include viral, bacterial and protozoal vaccines and their various components such as surface antigens. These include vaccines which contain glycoproteins, proteins or peptides derived from these proteins. Such vaccines are prepared from Staphylococcus aureus, Streptococcus pyogenes, Streptococcus pneumoniae, Neisseria meningitides, Neisseria gonorrhoeae, Salmonellae, Shigellae, Eschrichia coli, Klebsiellae, Proteus species, Vibrio cholerae, Helicobacterpylori, Pseudomonas aeruginosa, Haemophilus influenzae, Bordetella pertussis, Branhamella catarrhalis, Mycobacterium tuberculosis, Legionella pneumophila, Pneumocystis carinii, Treponema pallidum, Chlamydia, Telanus loxoid, Diphtheria toxoid, Influenza viruses, adenoviruses, paramyxo viruses rubella viruses, polioviruses, hepatitis viruses, herpes viruses, rabies viruses, HIV-1 viruses, HIV-2 viruses, and papilloma viruses. Other therapeutic proteins include those used for the treatment of autoimmune disease and to prevent transplant rejection. Yet other therapeutic proteins include those used for the treatment of cancer and influenza.

The present invention can also be utilized with Synagis®, Respigam, Synagis: CHD, CAOV-T (FluMist Liquid), HPV Cervical cancer vaccine; Epstein Barr Virus vaccine, CMV vaccine, Pneumococcal vaccine, hMPV/PIV-3/RSV vaccine, and Human metapneumovirus Mab.

In obtaining bacteria preparations, it is preferable to employ lyophilized bacteria which can be purchased. Alternatively, the bacteria can be grown, killed, washed and thereafter lyophilized.

Autoimmune disease is a disease in which the body produces an immunogenic response to some constituent of its own tissue. An autoimmune disease can be classified into those which predominantly affect one organ, such as hemolytic anemia and chronic thyroiditis, and those in which the autoimmune disease process is diffused through many tissues, such as multiple sclerosis, systemic lupus erythematosus, and arthritis. Exemplary autoimmune diseases and corresponding auto antigens include:

| Autoimmune Disease | Therapeutic Protein |
|---|---|
| Multiple Sclerosis | Myclin basic protein |
| Myasthemia Gravis | Acetyl choline receptor |
| Rheumatoid Arthritis | Type II collagen |
| Diabetes Mellitus | Insulin |
| Juvenile Diabetes Mellitus | Insulin |
| Autoimmune Thyroiditis | Thyroid proteins |

A second component which can be added to the therapeutic protein is a stabilizing agent. Stabilizing agents provide physical protection for the protein. Generally these stabilizing agents are therapeutically inactive water soluble sugars such as lactose, mannitol, and trehalose. These act to protect the therapeutic antigen during the coating process and the passage through the gastrointestinal tract.

The stabilizing medium ingredients can be present in a range of from about 1-10%, preferably at about 5%.

The antigen can be present in the range from about 0.5-10%, preferably at about 1%.

Antigen and Adjuvant can be present in a 1:2 ratio.

Adjuvant

The formulation also contains an adjuvant, although a single molecule may contain both adjuvant and antigen properties (e.g., cholera toxin) (Elson and Dertzbaugh, 1994). Adjuvants are substances that are used to specifically or non-specifically potentiate an antigen-specific immune response. Usually, the adjuvant and the formulation are mixed prior to presentation of the antigen but, alternatively, they may be separately presented within a short interval of time. For example the adjuvant can be pulsed once, twice or several times prior to antigen delivery or alternatively, the adjuvant can be pulsed in a variety of ways after the antigen is introduced.

Adjuvants include, for example, an oil emulsion (e.g., complete or incomplete Freund's adjuvant), a chemokine (e.g., defensins 1 or 2, RANTES, MIP1-.alpha., MIP-2, interleukin-8) or a cytokine (e.g., interleukin-1.beta., -2, -6, -10 or -12; gamma.-interferon; tumor necrosis factor-.alpha.; or granulocyte-monocyte-colony stimulating factor) (reviewed in Nohria and Rubin, 1994), a muramyl dipeptide derivative (e.g., murabutide, threonyl-MDP or muramyl tripeptide), a heat shock protein or a derivative thereof, a derivative of Leishmania major LeIF (Skeiky et al., 1995), cholera toxin or cholera toxin B, a lipopolysaccharide (LPS) derivative (e.g., lipid A or monophosphoryl lipid A), or superantigen (Saloga et al., 1996). Also, see Richards et al. (1995) for adjuvants useful in immunization.

An adjuvant may be chosen to preferentially induce antibody or cellular effectors, specific antibody isotypes (e.g., IgM, IgD, IgA1, IgA2, secretory IgA, IgE, IgG1, IgG2, IgG3, and/or IgG4), or specific T-cell subsets (e.g., CTL, Th1, Th2 and/or T.sub.DTH) (Glenn et al., 1995).

Cholera toxin is a bacterial exotoxin from the family of ADP-ribsoylating exotoxins (referred to as bAREs). Most bAREs are organized as A:B dimer with a binding B subunit and an A subunit containing the ADP-ribosyltransferase. Such toxins include diphtheria, Pseudomonas exotoxin A, cholera toxin (CT), E. coli heat-labile enterotoxin (LT), pertussis toxin, C. botulinum toxin C2, C. botulinum toxin C3, C. limosum exoenzyme, B. cereus exoenzyme, Pseudomonas exotoxin S, Staphylococcus aureus EDIN, and B. sphaericus toxin.

Cholera toxin is an example of a bARE that is organized with A and B subunits. The B subunit is the binding subunit and consists of a B-subunit pentamer which is non-covalently bound to the A subunit. The B-subunit pentamer is arranged in a symmetrical doughnut-shaped structure that binds to GM.sub.1-ganglioside on the target cell. The A subunit serves to ADP ribosylate the alpha subunit of a subset of the hetero trimeric GTP proteins (G proteins) including the Gs protein which results in the elevated intracellular levels of cyclic AMP. This stimulates release of ions and fluid from intestinal cells in the case of cholera.

Cholera toxin (CT) and its B subunit (CTB) have adjuvant properties when used as either an intramuscular or oral immunogen (Elson and Dertzbaugh, 1994; Trach et al., 1997). Another antigen, heat-labile enterotoxin from E. coli (LT) is 80% homologous at the amino acid level with CT and possesses similar binding properties; it also appears to bind the GM.sub.1-ganglioside receptor in the gut and has similar ADP-ribosylating exotoxin activities. Another bARE, Pseudomonas exotoxin A (ETA), binds to the .alpha..sub.2-macroglobulin receptor-low density lipoprotein receptor-related protein (Kounnas et al., 1992). bAREs are reviewed by Krueger and Barbieri (1995).

It is known in the art that cholera toxin (CT), its B subunit (CTB), E. coli heat-labile enterotoxin (LT), and pertussis toxin are potent adjuvants for transcutaneous immunization, inducing high levels of IgG antibodies but not IgE antibodies. It is also known that CTB without CT can also induce high levels of IgG antibodies. Thus, both bAREs and a derivative thereof can effectively immunize when epicutaneouly applied to the skin in a simple solution. As part of this invention it is apparent that pulsing the adjuvant would offer an advantage over static delivery so as to avoid possible toxic side effects elicited by the adjuvant.

When an adjuvant such as CT is mixed with BSA, a protein not usually immunogenic when applied to the skin, anti-BSA antibodies are induced. An immune response to diphtheria toxoid was induced using pertussis toxin as adjuvant, but not with diphtheria toxoid alone. Thus, bAREs can act as adjuvants for non-immunogenic proteins in an transcutaneous immunization system.

Protection against the life-threatening infections diphtheria, pertussis, and tetanus (DPT) can be achieved by inducing high levels of circulating anti-toxin antibodies. Pertussis may be an exception in that some investigators feel that antibodies directed to other portions of the invading organism are necessary for protection, although this is controversial (see Schneerson et al., 1996) and most new generation acellular pertussis vaccines have PT as a component of the vaccine (Krueger and Barbieri, 1995). The pathologies in the diseases caused by DPT are directly related to the effects of their toxins, and anti-toxin antibodies most certainly play a role in protection (Schneerson et al., 1996).

In general, toxins can be chemically inactivated to form toxoids which are less toxic but remain immunogenic. We envision that the transcutaneous immunization system using toxin-based immunogens and adjuvants can achieve anti-toxin levels adequate for protection against these diseases. The anti-toxin antibodies may be induced through immunization with the toxins, or genetically-detoxified toxoids themselves, or with toxoids and adjuvants such as CT. Genetically toxoided toxins which have altered ADP-ribosylating exotoxin activity, but not binding activity, are envisioned to be especially useful as non-toxic activators of antigen presenting cells used in transcutaneous immunization.

We envision that CT can also act as an adjuvant to induce antigen-specific CTLs through transcutaneous immunization (see Bowen et al., 1994; Porgador et al., 1997 for the use of CT as an adjuvant in oral immunization).

The bARE adjuvant may be chemically conjugated to other antigens including, for example, carbohydrates, polypeptides, glycolipids, and glycoprotein antigens. Chemical conjugation with toxins, their subunits, or toxoids with these antigens would be expected to enhance the immune response to these antigens when applied epicutaneously.

To overcome the problem of the toxicity of the toxins, (e.g., diphtheria toxin is known to be so toxic that one molecule can kill a cell) and to overcome the difficulty of working with such potent toxins as tetanus, several workers have taken a recombinant approach to producing genetically produced toxoids. This is based on inactivating the catalytic activity of the ADP-ribosyl transferase by genetic deletion. These toxins retain the binding capabilities, but lack the toxicity, of the natural toxins. This approach is described by Burnette et al. (1994), Rappuoli et al. (1995), and Rappuoli et al. (1996). Such genetically toxoided exotoxins could be useful for transcutaneous immunization system in that they would not create a safety concern as the toxoids would not be considered toxic. Additionally, several techniques exist to chemically toxoid toxins which can address the same problem (Schneerson et al., 1996). These techniques could be important for certain applications, especially pediatric applications, in which ingested toxins (e.g., diphtheria toxin) might possibly create adverse reactions.

Optionally, an activator of Langerhans cells may be used as an adjuvant. Examples of such activators include: inducers of heat shock protein; contact sensitizers (e.g., trinitrochlorobenzene, dinitrofluorobenzene, nitrogen mustard, pentadecylcatechol); toxins (e.g, Shiga toxin, Staph enterotoxin B); lipopolysaccharides, lipid A, or derivatives thereof; bacterial DNA (Stacey et al., 1996); cytokines (e.g., tumor necrosis factor-.alpha., interleukin-1.beta., -10, -12); and chemokines (e.g., defensins 1 or 2, RANTES, MIP-1.alpha., MIP-2, interleukin-8).

If an immunizing antigen has sufficient Langerhans cell activating capabilities then a separate adjuvant may not be required, as in the case of CT which is both antigen and adjuvant. It is envisioned that whole cell preparations, live viruses, attenuated viruses, DNA plasmids, and bacterial DNA could be sufficient to immunize transcutaneously. It may be possible to use low concentrations of contact sensitizers or other activators of Langerhans cells to induce an immune response without inducing skin lesions.

Adjuvant

The formulation of liposomes and antigen may also contain an adjuvant. Adjuvants are substances that are used to specifically or nonspecifically potentiate an antigen-specific immune response. Usually, the adjuvant and the formulation are mixed prior to presentation of the antigen but, alternatively, they may be separately presented within a short interval of time. Suitable adjuvants include, for example, an oil emulsion (e.g., complete or incomplete Freund's adjuvant), a chemokine (e.g., defensins 1 or 2, RANTES, interleukin-8) or a cytokine (e.g., interleukin-1, -2, -6, or -12; .gamma.-interferon; tumor necrosis factor; or granulocyte-monocyte-colony stimulating factor) (reviewed in Nohria and Rubin, 1994), a muramyl dipeptide derivative (e.g., murabutide, threonyl-MDP or muramyl tripeptide), a heat shock protein or a derivative, a derivative of Leishmania major LeIF (Skeiky et al., 1995), cholera toxin or cholera toxin B, or a lipopolysaccharide (LPS) derivative (e.g., lipid A or monophosphoryl lipid A). An adjuvant may be chosen to preferentially induce antibody or cellular effectors, specific antibody isotypes (e.g., IgM, IgD, IgA1, IgA2, secretory IgA, IgE, IgG1, IgG2, IgG3, and/or IgG4), or specific T-cell subsets (e.g., CTL, Th1, Th2 and/or T.sub.DTH) (Glenn et al., 1995).

Lipid A is derived from the lipopolysaccharide (LPS) of gram-negative bacterial endotoxin. It is an outstanding adjuvant that can be incorporated into the liposome bilayer to induce an immune response to a liposome-associated antigen (Alving, 1993). Lipid A is actually a heterogeneous mixture of compounds having similar structures (Banerji and Alving, 1979). The methods ordinarily used to obtain lipid A can produce a crude fraction, which is then purified by ethylenediamine tetraacetic acid and chloroform extraction to give a purified lipid A that is chloroform soluble (Banerji and Alving, 1979).

Other non-toxic adjuvants also fall into the type like aluminum hydroxide. (allergenics example).

The addition of bile salts in the form of bilosomes to oral vaccines has been reported to stimulate the potency of the immune response when added to synthetic measles peptide antigen-influenza vaccine combination therapy. (By Sharp, Acurian web site www.acurian.com) Similar penetration enhancers known to one skilled in the art of transdermal delivery may act as adjuvants. Those penetration enhancers known to disrupt the statum corneum, such as surfactants that may also be compatible with the antigen being delivered could be considered for this type of application.

Pulsing Schemes

Pulsatile delivery of the antigen/adjuvant combination can occur via various patterns of delivery. The following table outlines possible patterns of pulsing used to achieve optimum antigenic response. The table outlines the first several pulses in a system, but is not meant to indicate that the system is limited to 4 pulses. The pulsing patterns described are repeated until an appropriate therapeutic effect is attained either by titre or by previous experimentation and determination.

| Patch | | | | | Constant |
|---|---|---|---|---|---|
| Type | Pulse 1 | Pulse 2 | Pulse 3 | Pulse 4 | Delivery |
| I | Antigen | Adjuvant | Antigen | Adjuvant | |
| II | Adjuvant | Antigen | Adjuvant | Antigen | |
| III | Adjuvant | Adjuvant | Antigen | Adjuvant | Antigen |
| IV | Adjuvant | Adjuvant | Adjuvant | Adjuvant | Antigen |
| V | Antigen | Antigen | Antigen | Antigen | Adjuvant |

While not required for use with the transdermal method of the invention, permeability enhancers conventionally known in the art can also be present. Suitable permeability enhancers are listed but are not limited to fatty acid esters or fatty alcohol ethers of C₂₋₄ alkanediols, alcohols, such as ethanol, dimethyl sulfoxide, dimethyl lauramide, polyethylene glycol monolaurate and the like.

The dose and frequency of transdermal administration of a substance by the method of the invention depends on a number of factors, including the antigen/adjuvant combination being used, the intended use, potential skin irritation side effects, the lifetime of the substance, the tissue to which it is being administered, the age, weight and sex of any subject or patient. One skilled in the art will know how to evaluate these factors to determine a suitable dose and frequency of administration. A prior art rate control delivery device is designed to release an antigen/adjuvant at rates lower than that obtainable through skin of average permeability and to contain sufficient antigen/adjuvant such that unit activity is maintained throughout the desired rate of delivery.

Transdermal Delivery Devices and Mechanisms:

Numerous examples of transdermal delivery systems are know in the art and are currently on the market world wide. There are currently two types of transdermal drug delivery systems: “passive” and “active”. Passive systems deliver drug through the skin of the user unaided, which is the most popular type of transdermal system on the market. For specific indications though, Active systems are employed, which deliver the drug to or through the skin by use of an external mechanical device such as electricity, light, sonic force, heat or other herein unnamed method.

Possible ways to enable the technology in the scope of this invention would be through the use of iontophoresis, electrophoresis, electroosmosis, phonophoresis, sonophoresis, or otherwise un-named method of transdermal delivery method, such as silicon microneedle arrays (Remington: The science and practice of pharmacy, 20^{th} edition, 2002). In iontophoresis, electrical current is used to deliver ionic medicament to/through the skin. Iontophoretic devices are described in U.S. Pat. No. 4,820,263 (Spevak et al.), U.S. Pat. No. 4,927,408 ( Haak et al.), U.S. Pat. No. 5,084,008 (Phipps) and U.S. Pat. No. 6,377,847 (Keusch, et al.). The types of devices described in the references range from pastes, porous pads, cross-linked polymer supported devices, etc. Any of these types of devices can be adapted to pulse the antigen and adjuvant combination as described in the present invention.

Iontophoresis equipment is available from ISOKINETIcS, Inc. Therapy Equipment and Supplies. IOMED'S iontphoresis devices are easy to use, refillable and offer a reservoir system. Specific devices go by the trade name: TransQE, with Sur-Seal adhesive. Electrodes are also available from IOMED, and are compatible with Empi and Life-Tech units.

If one skilled in the art were determined to build their own reservoir units, numerous components for such devices can be obtained from various suppliers. The two suppliers for such materials listed here are not meant to be limiting in any way. The two suppliers are 3M, Minneapolis, Minn. and Berlex, Burlington, Vt. Specifically, 3M supplies CoTran backing, membranes and nonwoven backings. In addition 3M also offers a variety of foam tapes, Scotchpak Backings and Scotchpak Liners.

One can use emulsions as a medium or reservoir material to transdermally deliver the active agent, which protects the antigens until they reach the antigen presenting cells in the skin. This embodiment is particularly useful for delivery of active peptide fragments that require protection from degradation or additional assistance in traversing the layers of the skin. An emulsion is a dispersed system containing at least two immiscible liquid phases, a hydrophobic phase and a hydrophilic phase. The emulsion comprises the dispersed phase, the dispersion phase and an emulsifying agent or surfactant agent, except when the hydrophobic material is a “self-emulsifying” ester, whereby it is possible to produce an emulsion without a separate emulsifying agent. Usually one of the two immiscible liquids is an oil while the other is aqueous. Which phase becomes the dispersed phase depends on the relative amounts of the two liquid phases and which emulsifying agent is selected. Therefore, an emulsion in which the aqueous phase in the discontinuous phase is called a water-in-oil (w/o) emulsion and vice versa. The term “colloidal” refers to emulsions in which the dispersed phase is of very fine particles, usually less than about 1 mm in size. A “microcolloid” is an emulsion wherein the dispersed particles are usually about 300 .mu.m or less in size. Cosurfactants are also common components of microcolloids and are simply surfactants included in addition to the primary surfactant.

A “microemulsion” is an optically clear, isotropic and thermodynamically stable liquid. Microemulsions are composed of an oily phase, an aqueous phase, a surfactant, and sometimes, a cosurfactant. A homogenous mixture forms when components of the microemulsion are mixed together in any order. The resulting composition is thermodynamically stable with either a water continuous phase, an oily continuous phase, or a bicontinuous combination of the phases. Specifically, the microemulsion of the invention is a water-in-oil microemulsion, with the oil as the continuous phase.

Microemulsions are ideal for delivery of peptide fragment systems since they are homogenous, thermodynamically stable, have uniform droplet sizes of approximately 200-400 micrometers and are optically clear. A water-in-oil microemulsion, in particular, has small aqueous phase droplets, uniformly dispersed in a continuous oil phase. Therefore, the peptide is protected from proteolytic enzymes that may be present in the tissue. In general, the chemical structure of a peptide dictates that it will be at least somewhat, if not mostly, water soluble, and thus will be located inside the water droplet or very near the surface of the droplet of the water-in-oil microemulsion system. Thus, the outer oily phase of the microemulsion prohibits migration of proteolytic enzymes through the delivery system. The outer oily phase of the microemulsion is also able to incorporate into the intestinal cell matrix, thus creating membrane channels through which the peptide can pass. One general preparation procedure that maximizes peptide solubility is as follows: first, the peptide is prepared as a slurry in the aqueous phase at pH 2; second, the surfactant is added and mixed thoroughly; third, the oily phase is added and mixed to form the microemulsion.

The ingredients of the microemulsion can include any of the below named surfactants, oily phases or aqueous phases. The emulsions can either be macro- or microemulsions. Ordinary materials that are used to make emulsified hydrophobic and hydrophilic phases are contemplated. These materials include, but are not limited to, surfactants, aqueous and nonaqueous hydrophilic materials and numerous hydrophobic materials. Non-limiting examples of surfactants are polyoxyethylene sorbitan esters, ethyleneoxide propylene oxide block copolymers, polyglycolized glycerides, sucrose esters, polyoxyethylene laurel esters, and others. Non-limiting examples of hydrophilic materials are various aqueous buffered systems, polyethylene glycols, diethylene glycol monoethyl ether, and others. Non-limiting examples of hydrophobic materials are carboxylic acid esters, fatty acids, glyceryl derivatives such as glyceryl behenate, short, medium and long chain triglycerides and others.

Macro or gross emulsions can be made by conventional emulsion methods. In large scale manufacture, these steps can be accomplished using standard mixing equipment employed in the production of ointments, creams and lotions. Specifically, mixing tanks made by Lee Industries (New Cumberland, Pa.) can be readily used. Regardless of the equipment employed, mixing needs to be accomplished using as low a shear rate as practical, in order to maintain the physical integrity of the peptide. The bioactive agent can be added to the cooled mixture at a suitable temperature for stability and activity purposes. Microemulsions, which are spontaneously formed, isotopically clear liquids are formed with mechanical mixing of the ingredients. The bioactive agent can be added either to the hydrophilic phase prior to mixing with the surfactant and hydrophobic phases or after the microemulsion is formed, depending on stability and activity of the bioactive agent. The emulsions can be filled into hard or soft gelatin capsules and optionally coated with enteric polymers.

The incorporated peptide/protein is further protected from peptidases and proteases with the addition of a hydrophobic thickening agent in the oily phase. An additional hydrophobic ingredient, when added to the microemulsion, forms a paste-like composition that becomes liquefied at about 37.degree. C.

Monitoring Antigenic Activity:

It is understood that individuals receiving immunization may also be receiving additional preventative treatment for bacterial or viral infection, such as strepto pneumonococus or influenza.

It will be appreciated that unit content of active ingredient(s), whole plasma proteins or their active fragment(s) or analog(s), contained in an individual dose of each dosage form need not in itself constitute an effective amount, since the necessary effective amount can be reached by administration of a plurality of dosage units (such as by repeated pulsing). Administration of an effective dosage may be in a single dose form or in multiple dosage forms and it may be provided with an enteric coating and/or a sustained release mechanism, such as a reservoir.

As is typical from the traditional means of antigen delivery, and from the newer transdermal method, the antigenic activity resulting from the delivery of antigen with and without an adjuvant is typically monitored by measuring the antibody level in plasma initially and 2-4 weeks subsequent to the application of the antigen. As expected, several applications of the antigen may be necessary to confer immunity. U.S. Pat. No. 5,980,898 (Glenn et al.) and U.S. Pat. No. 5,910,306 (Alving et al.) have described monitoring the results of immunization and boosting immunization in the examples, those patents are hereby incorporated by reference in their entireties. It would be understood that this type of work is both customary and necessary in order to determine an effective application of the antigen.

### EXAMPLES

The following examples are provided to illustrate the invention and should not be regarded as limiting the invention in any way.

### Example 1

Allergenic protein such as oak pollen

Adjuvant such as aluminium hydroxide

Aqueous medium with stabilizing agent

Package in a transdermal device with an appropriate mechanism, such as an IOMED complete iontophoresis set up containing the Trans QE delivery system to which the dosage form is added to allow the contents to come in contact with the skin. The patch will be equipped with leads to which electrodes can be attached to effect electrotransport. In the case that the medicament to be delivered to the skin is a cation, the reservoir is connected to the electrode which acts as an anode. The return electrode would act as a cathode. If the medicament is an anion, then the drug containing reservoir would be connected to the cathode and the return reservoir would be connected to the anode. The reservoir containing the adjuvant would also be connected to electrodes in a similar manner, with an independent pulsing device.

To accomplish the delivery of antigen/adjuvant to the skin, a sequence of electrical pulses (between 20 and 200V peak to peak, preferably, and between 10 and 15,000 Hz is preferably provided to the electrodes that are placed in contact with the delivery device.

Specifically, an electrical burst of pulses at 2,200 Hz are provided to the skin at a burst ON/OFF frequency e.g., 50 Hz by way of an electrode array. Electrical pulses are provided by a pulse generator, in which a transformer is used as an element of the pulse generator. Several iontophoresis electrical generators are currently available in the market, either D.C. or D.C. pulsed.

### Example 2

streptococcus pneumonae vaccine

Adjuvant

Administer pulse transdermal system, as in Example 1, at time 0, 1 year, and thereafter as determined by necessary by titre.

### Example 3

herpes vaccine

Adjuvant

Administer pulse transdermal system, as in Example 1, at time 0, and monthly, or once daily following periods of high stress or sun exposure.

### Example 4

HIV

Adjuvant

Administer pulse transdermal system, as in Example 1, on a weekly or monthly basis as determined by healthcare professional.

### Example 5

Influenza

Adjuvant

Administer pulse transdermal system, as in Example 1, prior to flu season

### Example 6

H. Pylori

Adjuvant

Administer pulse transdermal system, as in Example 1, weekly until breath test is negative.

### Example 7

Therapeutic protein such as human papillomavirus vaccine (or other cancer vaccine)

Adjuvant such as Aluminum

Stabilizing medium

Apply to skin using a reservoir system known to one skilled in the art of transdermal delivery. Transdermal delivery device can be single or double compartment. In the case of a single compartment, then only one set of electrophoresis leads is present. In the case of a double compartment device, then two sets of electrophoresis leads are present and one can affect the desired pattern of pulsing as described in Example 1.

### Example 8

Autoimmune disease antigen

Adjuvant such as Aluminum

Stabilizing medium

Apply to skin using a reservoir system known to one skilled in the art of transdermal delivery. Transdermal delivery device can be single or double compartment. In the case of a single compartment, then only one set of electrophoresis leads is present. In the case of a double compartment device, then two sets of electrophoresis leads are present and one can affect the desired pattern of pulsing as described in Example 1.

We claim:

1. A method for triggering an immunogenic response to an immunogen in a patient or subject comprising: transdermally delivering an antigen to the Langerhans cells of the skin, whereby at least a portion of said transdermal delivery occurs in pulses.

2. A method for triggering an immunogenic response to an immunogen in a patient or subject comprising: transdermally delivering an antigen and an adjuvant to the Langerhans cells of the skin, whereby at least a portion of said transdermal delivery occurs in pulses.

3. A method for eliciting an improved immunogenic response to an immunogen in a patient or subject comprising: transdermally delivering an antigen to the Langerhans cells of the skin, whereby at least a portion of said transdermal delivery occurs in pulses.

4. A method for eliciting an improved immunogenic response to an immunogen in a patient or subject comprising: transdermally delivering an antigen and an adjuvant to the Langerhans cells of the skin, whereby at least a portion of said transdermal delivery occurs in pulses.

5. The method of claim 1, 2, 3, or 4, wherein the antigen is delivered in pulses.

6. The method of claim 2 or 4, wherein the adjuvant is delivered in pulses.

7. The method of claim 1, 2, 3, or 4, wherein the antigen is delivered in a continuous bolus.

8. The method of claim 2 or 4, wherein the adjuvant is delivered in a continuous bolus.

9. The method of claim 1, 2, 3, or 4, wherein said delivery lasts between 1 to 3 hours.

10. The method of claim 1, 2, 3, or 4, wherein each of said pulses lasts between 1 to 30 minutes.

11. A method for treating herpes attacks in a patient or subject comprising: applying the method of claim 1, 2, 3, or 4 every four hours for two days.

12. A method for treating allergy attacks in a patient or subject comprising: applying the method of claim 1, 2, 3, or 4 for two hours once every three weeks.

13. A method for treating cancer in a patient or subject comprising: applying the method of claim 1, 2, 3, or 4 for 1 to 8 hours over a period of from 1 to 7 days at intervals of from 1 to 3 months extending over a period of from 1 to 3 years.
